(19) United States
(12) Patent Application Publication
(10) Pub. No.: US 2022/0257688 A1
(43) Pub. Date: Aug. 18, 2022

(54) COMPOSITION COMPRISING SALVIA MILTIORRHIZA OR PAEONIA LACTIFLORA EXTRACT AS ACTIVE INGREDIENT FOR PREVENTION OR TREATMENT OF LIPID METABOLISM DISORDER

(71) Applicant: USCAREPHARM CO.,LTD, Suwon-si, Gyeonggi-do (KR)

(72) Inventors: Suk Jin SONG, Yongin-si, Gyeonggi-do (KR); Byulnim OH, Seoul (KR); Mi-Ran CHA, Yongin-si, Gyeonggi-do (KR); Sunhee KANG, Yongin-si, Gyeonggi-do (KR); Juah SON, Yongin-si, Gyeonggi-do (KR); Yunsung LEE, Seoul (KR)

(73) Assignee: USCAREPHARM CO.,LTD, Suwon-si, Gyeonggi-do (KR)

(21) Appl. No.: 17/628,235

(22) PCT Filed: Aug. 12, 2020
(86) PCT No.: PCT/KR2020/010684

§ 371 Date: Jan. 19, 2022

(30) Foreign Application Priority Data
Aug. 16, 2019 (KR) 10-2019-0100433

(51) Int. Cl. A61K 36/65; A61K 36/537; A61P 3/06

(52) U.S. Cl. CPC A61K 36/65; A61K 36/537; A61K 2236/333; A61K 2236/331; A61P 3/06

(57) ABSTRACT

The present invention relates to a composition comprising a Salvia miltiorrhiza or Paeonia lactiflora extract as an active ingredient for prevention or treatment of lipid metabolism disorder and, more particularly, provides a pharmaceutical composition and a health functional food composition, each comprising a Salvia miltiorrhiza extract, a Paeonia lactiflora extract, or an extract from a mixture of Salvia miltiorrhiza and Paeonia lactiflora as an active ingredient for prevention or treatment of lipid metabolism disorder. The composition prevents the differentiation of preadipocyte cells to adipocyte cells, reduces blood triglyceride and cholesterol levels, and effectively suppresses fatty liver and visceral fat formation and as such, can be advantageously used as a pharmaceutical and food composition for prevention, alleviation, or treatment of lipid metabolism disorder.

### TECHNICAL FIELD

The present invention relates to a composition containing a Salvia miltiorrhiza or Paeonia lactiflora extract as an active ingredient for the prevention or treatment of lipid metabolism disorders.

### BACKGROUND ART

With the introduction of Western diets, as the intake of calories through animal foods and processed foods increases, the incidence of adult diseases is increasing. In particular, high triglycerides and high cholesterol in the blood due to excessive intake of animal fat are considered to be important causes for the onset of chronic adult diseases, and it has been reported that triglycerides are the cause of heart disease and obesity. Triglycerides are also one of the causes of metabolic syndrome, which is represented by abdominal obesity, hypertension, hypertriglyceridemia, hyperglycemia, and hyperlipidemia.

Triglyceride, commonly referred to as neutral fat, is a type of lipid, is a representative storage fat in which three fatty acid molecules are combined with one glycerin molecule, and accounts for 95% of body fat. Recently, the triglyceride problem is so serious that about one-third of Koreans have a blood triglyceride concentration of 150 md/dL.

In particular, abdominal obesity is closely related to triglycerides. Abdominal obesity is caused by an increase in the number of adipocytes in the mesentery, and adipocytes in the mesentery are easily digested, so the decomposed fat flows into the geographically nearby liver or is accumulated as fat in the abdomen. Fatty acids introduced into the liver are discharged into the blood to cause hypertriglyceridemia or increase blood sugar, so abdominal obesity and fatty liver are closely related to blood triglyceride levels.

Lipid is a concept that encompasses cholesterol in addition to triglycerides, and common lipid tests include triglycerides, total cholesterol, low-density lipoprotein (LDL) cholesterol, and high-density lipoprotein (HDL) cholesterol. LDL cholesterol is also known as ‘bad cholesterol, and high LDL cholesterol levels increase the risk of heart attack and stroke. HDL cholesterol is also known as ‘good cholesterol, and high HDL cholesterol levels reduce the risk of heart attack or stroke. Diseases in which triglycerides, total cholesterol, LDL cholesterol are increased or HDL cholesterol is decreased are referred to as hyperlipidemia, hypercholesterolemia, hypertriglyceridemia, and the like. Typically, dyslipidemia is a disease name encompassing the above diseases.

Dyslipidemia is diagnosed if an abnormality is found in at least one item in the case that the following items are measured twice or more. Reference levels of total cholesterol below 200 mg/dL, LDL cholesterol below 130 mg/dL, HDL cholesterol below 60 mg/dL, and triglyceride below 150 mg/dL indicate target values for those without risk factors such as heart disease or stroke. Normal ranges for people with risk factors may be different.

Treatment of dyslipidemia includes taking alone or in combination drugs such as statins, fibrates, nicotinic acids, Ezetimibe, and Omega-3 fatty acids. However, statins may cause hepatotoxicity and myopathy, and fibrates may cause gallstones. Nicotinic acids may cause skin flushing, digestive disorders, hepatotoxicity, gout, etc., and , Ezetimibe may increase intestinal gas and cause constipation. In addition, omega-3 fatty acids are not only less convenient to take due to fishy smell and high doses, but also have serious side effects on drugs currently used, such as skin rashes, so there is a need for a safer and more effective therapeutic agent.

### DISCLOSURE

### Technical Problem

In order to solve the above problems, the present invention provides a pharmaceutical composition and a health functional food composition containing a natural product-derived extract as an active ingredient for the prevention or treatment of lipid metabolism disorders.

In addition, the present invention provides a health functional food composition for alleviating blood lipids, containing a natural product-derived extract as an active ingredient.

### Technical Solution

A pharmaceutical composition for prevention or treatment of lipid metabolism disorders according to the present invention may contain a Salvia miltiorrhiza extract, a Paeonia lactiflora extract, or a mixed extract of Salvia miltiorrhiza and Paeonia lactiflora as an active ingredient.

Health functional food composition for prevention or alleviation of lipid metabolism disorders according to the present invention may contain a Salvia miltiorrhiza extract, a Paeonia lactiflora extract, or a mixed extract of Salvia miltiorrhiza and Paeonia lactiflora as an active ingredient.

Health functional food composition for alleviation of blood lipids according to the present invention may contain a Salvia miltiorrhiza extract, a Paeonia lactiflora extract, or a mixed extract of Salvia miltiorrhiza and Paeonia lactiflora as an active ingredient.

### Advantageous Effects

Since a Salvia miltiorrhiza extract, a Paeonia lactiflora extract, or a mixed extract of Salvia miltiorrhiza and Paeonia lactiflora according to the present invention prevents differentiation of preadipocytes into adipocytes, reduces blood triglyceride and cholesterol levels, and effectively suppresses fatty liver and visceral fat formation, it can be advantageously used as a pharmaceutical and food composition for prevention, alleviation, or treatment of lipid metabolism disorders.

In addition, the extract can prevent, alleviate or treat lipid metabolism disorders safely and effectively without side effects by using natural products.

### BRIEF DESCRIPTIONS OF THE DRAWINGS

FIG. 1 is a graph showing the effects of inhibiting accumulation of fat in hepatocytes according to treatment with a Salvia miltiorrhiza or Paeonia lactiflora extract, or mixed extracts of Salvia miltiorrhiza and Paeonia lactiflora, according to an experimental example of the present invention.

FIG. 2 is a graph showing changes in triglyceride (TG) content according to treatment with a Salvia miltiorrhiza or Paeonia lactiflora extract, or mixed extracts of Salvia miltiorrhiza and Paeonia lactiflora, according to an experimental example of the present invention.

FIG. 3 shows graphs showing changes in cholesterol content according to treatment with a Salvia miltiorrhiza or Paeonia lactiflora extract, or mixed extracts of Salvia miltiorrhiza and Paeonia lactiflora according to an experimental example of the present invention, wherein A shows total cholesterol content, B shows low-density lipoprotein (LDL) cholesterol content, and C shows high-density lipoprotein (HDL) cholesterol content.

FIG. 4 shows histopathological examination results of a mixed extract according to an experimental example of the present invention.

### BEST MODE

Hereinafter, the present invention will be described in detail. The present inventors searched for natural product-derived materials that effectively prevent or treat diseases caused by blood lipids accumulation or diseases caused by adipocytes differentiation by effectively reducing blood lipids without side effects, and 5 kinds of materials were selected from the library of 100 kinds of natural product extracts. Among them, Salvia miltiorrhiza and Paeonia lactiflora with the best effects were selected as the final materials, and as a result of testing effects of an extract from a mixture of two kinds of finally selected herbal medicines, the present invention was completed by determining that an extract from a mixture of Salvia miltiorrhiza and Paeonia lactiflora was effective in improving blood lipids by inhibiting the adipocytes differentiation and generation of triglycerides and cholesterol in vitro or in vivo.

In the present invention, “Salvia miltiorrhiza” is a herbaceous perennial plant belonging to the family Lamiaceae and is called Salvia miltiorrhiza because it resembles the shape of Ginseng and has a red color. The dried root of Salvia miltiorrhiza has been traditionally used to improve blood circulation and treat congestion and is also called Geukseoncho, Mokyangyu, Bunmacho, Red Ginseng, or Honggeun.

In the present invention, “Paeonia lactiflora” is a perennial herb belonging to the family Paeoniaceae with strong cold resistance, and grows in the low mountains of the central and northern regions. It has been cultivated for ornamental and medicinal purposes since ancient times, its root is used as a medicine in Oriental medicine. Paeonia obovata Max. the root is used as an anemia treatment and analgesic, and Paeonia lactiflora Pall root is used as a blood pressure-lowering agent and antipyretic agent.

In the present invention, the term “prevention” means any action that inhibits the occurrence or delays the onset of a lipid metabolism disorder or at least one symptom of the lipid metabolism disorder by administration of the pharmaceutical composition or health functional food composition according to the present invention. It also includes treatment of a subject in remission of the disease to prevent or inhibit recurrence.

In the present invention, the term “treatment” means any action that improves or beneficially changes at least one symptom of the lipid metabolism disorder, for example, relieves, reduces, or eliminates the lipid metabolism disorder or the symptom by administering the pharmaceutical composition according to the present invention.

In the present invention, the term “alleviation” refers to any action that improves or beneficially changes at least one symptom of the lipid metabolism disorder, for example, relieves, reduces, or eliminate the lipid metabolism disorder or the symptom by ingestion of the health functional food composition according to the present invention.

In the present invention, “triglyceride (TG)” is a type of lipid, and may be used interchangeably with “neutral fat” The energy that is not used in the body is accumulated as subcutaneous fat, and most of it is accumulated as triglycerides, which, along with cholesterol, can act as a major cause of arteriosclerosis, diabetes, obesity, and the like.

The present invention provides a pharmaceutical composition for prevention or treatment of lipid metabolism disorder, containing a Salvia miltiorrhiza extract, a Paeonia lactiflora extract, or a mixed extract of Salvia miltiorrhiza and Paeonia lactiflora as an active ingredient.

In the pharmaceutical composition according to the present invention, the Salvia miltiorrhiza extract, the Paeonia lactiflora extract, or the mixed extract of Salvia miltiorrhiza and Paeonia lactiflora may be extracted with anyone solvent selected from the group consisting of water, alcohols having 1 to 4 carbon atoms, and mixed solvents thereof, and preferably, may be extracted with 60 to 80 wt % ethanol aqueous solutions, more preferably a 70 wt % ethanol aqueous solution, but the present invention is not limited thereto. According to an experimental example of the present invention, the extraction yield of Salvia miltiorrhiza and Paeonia lactiflora was the highest for the 70 wt % ethanol aqueous solution, and the triglyceride inhibitory effect and adipocytes differentiation inhibitory effect were also the most excellent.

As the extraction method, any one of methods such as hot water extraction, cold extraction, reflux cooling extraction, solvent extraction, steam distillation, ultrasonic extraction, elution, and compression may be selected and used. In addition, the desired extract may be further subjected to a conventional fractionation process and may be purified using a conventional purification method.

There is no limitation on the method for preparing the extract, and any known method may be used. For example, the extract may be prepared in a powder state by an additional process such as distilling under reduced pressure and freeze-drying or spray-drying the primary extract extracted by the hot water extraction or solvent extraction method described above. In addition, it is also possible to obtain fractions further purified by using various chromatography methods such as silica gel column chromatography, thin-layer chromatography, and high-performance liquid chromatography for the primary extract. Therefore, in the present invention, the mixed extract of Salvia miltiorrhiza and Paeonia lactiflora is a concept including all extracts, fractions, and purified products, their dilutions, concentrates, or dried products obtained in each step of extraction, fractionation, or purification.

According to one preparation example of the present invention, the extract is obtained by extraction by reflux cooling method using alcohol (ethanol) as an extraction solvent and concentration under reduced pressure and dry, the extraction solvent and extraction method for extracting active ingredients of Salvia miltiorrhiza and Paeonia lactiflora are not limited thereto.

The pharmaceutical composition according to the present invention may contain a single extract of the Salvia miltiorrhiza extract or the Paeonia lactiflora extract as an active ingredient and may contain a mixed extract of Salvia miltiorrhiza and Paeonia lactiflora as an active ingredient. The mixed extract may be extracted from a mixture of Salvia miltiorrhiza and Paeonia lactiflora in a weight ratio of (0.1 to 10): 1, preferably (0.2 to 5): 1, and more preferably 1:1. According to an experimental example of the present invention, it was found that the triglyceride inhibitory effect and adipocytes differentiation inhibitory effect were the most excellent in the extract from the mixture of Salvia miltiorrhiza and Paeonia lactiflora in the weight ratio of 1:1.

Accordingly, in the pharmaceutical composition according to the present invention, the mixed extract of Salvia miltiorrhiza and Paeonia lactiflora may be extracted from a mixture of Salvia miltiorrhiza and Paeonia lactiflora in the weight ratio of 1:1 with a 70 wt % ethanol aqueous solution.

The pharmaceutical composition according to the present invention can prevent the differentiation of preadipocytes into adipocytes, suppress the accumulation of triglycerides in preadipocytes or hepatocyte cells, and reduce blood triglycerides or blood cholesterol. In addition, it can effectively suppress fatty liver and visceral fat formation. According to one experimental example of the present invention, it was found that a Salvia miltiorrhiza extract, a Paeonia lactiflora extract, or a mixed extract of Salvia miltiorrhiza and Paeonia lactiflora extract effectively reduced blood triglycerides, total cholesterol, and LDL cholesterol content and increased HDL cholesterol content in animal experiments. In addition, it was found that the mixed extract of Salvia miltiorrhiza and Paeonia lactiflora effectively reduced fatty liver and visceral fat in animal experiments. Due to these effects, the composition can be used as a pharmaceutical composition for the prevention or treatment of a lipid metabolism disorder. The lipid metabolism disorder may be one or more disorders selected from the group consisting of dyslipidemia, fatty liver, and obesity, but is not limited thereto.

Dyslipidemia is a state in which blood lipids are increased or decreased than normal, more specifically, a state in which total cholesterol, LDL cholesterol, or triglycerides in the blood are increased or HDL cholesterol is decreased. It may include hypertriglyceridemia, hypercholesterolemia, or hyperlipidemia, but is not limited thereto. Hypertriglyceridemia refers to a state in which triglycerides are increased in the blood, and hypercholesterolemia is a state in which cholesterol is increased in the blood, and total cholesterol and LDL cholesterol are high. In addition, hyperlipidemia is a state in which lipids including cholesterol and triglycerides are increased in the blood, thereby increasing the risk of coronary artery disease such as arteriosclerosis or myocardial infarction.

The fatty liver is a reversible disease in which excess fat, mainly triglyceride, is accumulated in the liver and increases in fat content, and may appear together with diseases such as hyperlipidemia.

In addition, obesity may include only abdominal obesity associated with visceral fat accumulation.

The visceral fat is a tissue composed of adipocytes in vivo and is also referred to as white adipose tissue. White adipose tissue is distributed in the subcutaneous and the retroperitoneal cavity and is a tissue that stores energy in the form of triglycerides that accounts for more than 80% of adipocytes.

The pharmaceutical composition according to the present invention may further include an adjuvant in addition to the extract. The adjuvant may be used without limitation as long as it is known in the art, for example, Freund's complete adjuvant or incomplete adjuvant may be further included to increase the effect.

The pharmaceutical composition according to the present invention may be prepared in a form in which the active ingredient is incorporated into a pharmaceutically acceptable carrier. Here, the pharmaceutically acceptable carriers include carriers, excipients, and diluents commonly used in the pharmaceutical field. Pharmaceutically acceptable carriers that can be used in the pharmaceutical composition of the present invention may include but are not limited to, lactose, dextrose, sucrose, sorbitol, mannitol, xylitol, erythritol, maltitol, starch, gum acacia, alginate, gelatin, calcium phosphate, calcium silicate, cellulose, methyl cellulose, polyvinyl pyrrolidone, water, methyl hydroxybenzoate, propyl hydroxybenzoate, talc, magnesium stearate, and mineral oil.

The pharmaceutical composition of the present invention may be formulated and used in the form of oral dosage forms such as powders, granules, tablets, capsules, suspensions, emulsions, syrups, and aerosols, external preparations, suppositories, or sterile injection solutions according to conventional methods, respectively.

In the case of formulation, it may be prepared using a diluent or excipient such as a filler, extender, binder, wetting agent, disintegrant, and surfactant commonly used. Solid formulations for oral administration include tablets, pills, powders, granules, capsules, and the like, and such solid formulations may be prepared by mixing the active ingredient with at least one excipient, for example, starch, calcium carbonate, sucrose, lactose, and gelatin. In addition to simple excipients, lubricants such as magnesium stearate and talc may also be used. Liquid formulations for oral administration include suspensions, solutions, emulsions, syrups, and the like. In addition to water and liquid paraffin, which are commonly used diluents, various excipients such as wetting agents, sweeteners, fragrances, and preservatives may be included.

Formulations for parenteral administration include sterile aqueous solutions, non-aqueous solutions, suspensions, emulsions, lyophilized formulations, and suppositories. As the non-aqueous solvent and suspending agent, propylene glycol, polyethylene glycol, vegetable oil such as olive oil, and injectable ester such as ethyl oleate may be used. As the base of the suppository, witepsol, tween 61, cacao butter, laurin, glycerogelatin, and the like may be used.

The pharmaceutical composition according to the present invention may be administered to an individual by various routes. All modes of administration may be envisaged, for example, by oral, intravenous, intramuscular, subcutaneous, and intraperitoneal injection.

The dosage of the pharmaceutical composition according to the present invention is selected in consideration of the individual's age, weight, sex, physical condition, and the like. In the pharmaceutical composition according to the present invention, it is self-evident that the concentration of the extract can be variously selected depending on the subject, and the concentration is preferably included in the pharmaceutical composition at a concentration of 0.01˜5,000 μg/ml. In the case that the concentration is less than 0.01 μg/ml, pharmaceutical activity may not appear, and in the case that it exceeds 5,000 μg/ml, it may be toxic to the human body.

The pharmaceutical composition according to the present invention may be used alone for the prevention or treatment of lipid metabolism disorders or may be used in combination with surgery or other drug treatment.

The present invention provides a health functional food composition for prevention or alleviation of lipid metabolism disorders, containing a Salvia miltiorrhiza extract, a Paeonia lactiflora extract, or a mixed extract of Salvia miltiorrhiza and Paeonia lactiflora as an active ingredient.

In addition, the present invention provides a health functional food composition for alleviation of blood lipids, containing a Salvia miltiorrhiza extract, a Paeonia lactiflora extract, or a mixed extract of Salvia miltiorrhiza and Paeonia lactiflora as an active ingredient.

In the health functional food composition according to the present invention, the Salvia miltiorrhiza extract, the Paeonia lactiflora extract, or the mixed extract of Salvia miltiorrhiza and Paeonia lactiflora may be extracted with anyone solvent selected from the group consisting of water, alcohols having 1 to 4 carbon atoms, and mixed solvents thereof, preferably, with a 60 to 80 wt % ethanol aqueous solution, and more preferably with a 70 wt % ethanol aqueous solution, but the present invention is not limited thereto.

The health functional food composition according to the present invention may contain a single extract of the Salvia miltiorrhiza extract or the Paeonia lactiflora extract as an active ingredient and may contain a mixed extract of Salvia miltiorrhiza and Paeonia lactiflora as an active ingredient. The extract may be extracted from a mixture of Salvia miltiorrhiza and Paeonia lactiflora in a weight ratio of (0.1 to 10): 1, preferably (0.2 to 5): 1, and more preferably 1:1.

Accordingly, in the health functional food composition according to the present invention, the mixed extract may be extracted from a mixture of Salvia miltiorrhiza and Paeonia lactiflora in the weight ratio of 1:1 with the 70 wt % ethanol aqueous solution.

The health functional food according to the present invention can inhibit the differentiation of preadipocytes into adipocytes, inhibit the accumulation of triglycerides in preadipocytes or hepatocytes, and reduce blood triglycerides or blood cholesterol. In addition, it can effectively suppress fatty liver and visceral fat formation. Due to these effects, the composition can be used as a health functional food composition for the prevention or alleviation of lipid metabolism disorders, or a health functional food composition for alleviation of blood lipids. The lipid metabolism disorder may be one or more diseases selected from the group consisting of dyslipidemia, fatty liver, and obesity, but is not limited thereto.

The health functional food composition according to the present invention may be manufactured and processed in the form of tablets, capsules, powders, granules, liquids, and pills to prevent or alleviate lipid metabolism disorders and alleviate blood lipids. In the present invention, the term ‘health functional food composition’ refers to food manufactured and processed using raw materials or ingredients having useful functions in the human body according to the Health Functional Foods Act No. 6727, and it is taken to obtain useful effects for health purposes, such as regulating nutrients or physiological effects for the structure and function of the human body.

The health functional food composition according to the present invention may contain various flavoring agents or natural carbohydrates as additional ingredients like a conventional food composition in addition to the extract as an active ingredient. Examples of natural carbohydrates include monosaccharides such as glucose, and fructose; disaccharides such as maltose and sucrose; polysaccharides, conventional sugars such as dextrin and cyclodextrin, and sugar alcohols such as xylitol, sorbitol, and erythritol. As the above-mentioned flavoring agents, natural flavoring agents (Taumatine), stevia extract (eg rebaudioside A, glycyrrhizin, etc.), and synthetic flavoring agents (saccharin, aspartame, etc.) may be advantageously used.

The health functional food composition according to the present invention may be formulated in the same manner as the pharmaceutical composition and used as a functional food or added to various foods. Foods to which the composition according to the present invention can be added may include, for example, beverages, meat, chocolate, foods, confectionery, pizza, ramen, other noodles, gums, candy, ice cream, alcoholic beverages, vitamin complexes, and health supplements.

In addition, the composition may contain various nutrients, vitamins, minerals (electrolytes), flavoring agents such as synthetic flavoring agents and natural flavoring agents, colorants, and thickeners (cheese, chocolate, etc.), pectic acid and salts thereof, alginic acid, and salts thereof, organic acids, protective colloid thickeners, pH adjusters, stabilizers, preservatives, glycerin, alcohols, and carbonation agents used in carbonated beverages, in addition to the extract as the active ingredient. In addition, the composition may contain natural fruit juice and pulp for the production of fruit juice beverages and vegetable beverages.

The health functional food according to the present invention may contain normal food additives, and unless otherwise regulated, the suitability as a food additive is determined according to the standards and criteria for the relevant item by the general rules and general test methods of the Food Additives Code approved by the Ministry of Food and Drug Safety. The items listed in the ‘Food Additives Code’ include, for example, chemical compounds such as ketones, glycine, calcium citrate, nicotinic acid, and cinnamic acid; natural additives such as persimmon pigment, licorice extract, crystalline cellulose, high pigment, and guar gum; and mixed formulations, such as a sodium L-glutamate formulation, a noodle-added alkali agent, a preservative formulation, and a tar color formulation.

For example, the health functional food in the form of tablets may be formed by granulating conventionally a mixture obtained by mixing the active ingredient of the present invention, such as the Salvia miltiorrhiza extract, the Paeonia lactiflora extract, or the mixed extract of Salvia miltiorrhiza and Paeonia lactiflora, with excipients, binders, disintegrants, and other additives, followed by compression molding of the mixture by adding a lubricant and the like or direct compression molding. In addition, health functional food in the form of tablets may contain flavors, if necessary. Among health functional foods in the form of capsules, hard capsules may be prepared by filling a mixture of the extract with additives such as excipients in a conventional hard capsule, and soft capsules may be prepared by filling a mixture of the extract with additives such as excipients in a capsule base such as gelatin. The soft capsules may contain plasticizers such as glycerin or sorbitol, colorants, or preservatives, if necessary. The health functional food in the form of pills may be prepared by molding a mixture of the extract and an excipient, a binder, a disintegrant, etc. by a known method, and if necessary, sucrose or another coating agent may be applied to the pills or the surface of the pills may be coated with a material such as starch or talc. The health functional food in the form of granules may be prepared in a granular form of a mixture of the extract and excipients, binders, or disintegrants by a conventionally known method and may contain flavoring agents or flavors, as necessary.

The composition has the advantage that, unlike general drugs, there are no side effects that may occur during long-term administration of the drugs by using food as raw material, has excellent portability, and can be ingested as an adjuvant for prevention or alleviation of lipid metabolism disorders and improvement of blood lipids.

### MODES FOR CARRYING OUT INVENTION

Hereinafter, examples will be described in detail to help the understanding of the present invention. However, the following examples are merely illustrative of the present invention, and the scope of the present invention is not limited to the following examples. The examples of the present invention are provided to more completely explain the present invention to those of ordinary skill in the art.

### <PREPARATION EXAMPLE 1>PREPARATION OF RAW MATERIAL EXTRACT

Single or mixed extraction conditions of each medicine; according to the conditions of alcohol (ethanol) concentration (0, 20, 50, 70, and 100%), extracts were prepared. More specifically, first, each herbal medicine was mixed according to each condition by weight ratio and extracted, by using water or 20 to 100% alcohol selected as an extraction solvent according to each condition. The temperature of the extract was maintained at 70° C. using a multi-heating mantle (WiseTherm), the reflux cooling device was connected to a low-temperature constant temperature water tank (A&D), and the extract extracted under reflux cooling were concentrated under reduced pressure in a water bath at 45° C. and a pressure of 200 mbar or less using a vacuum rotary concentrator (EYELA) to finally obtain extracts of each condition. Then, the yield (%) of the extract according to each of the above conditions was checked.

### <Experimental Example 1> Determination of the Effect of Reducing Triglycerides (TG) Content in Preadipocytes According to Single or Mixed Extracts

Experiments were performed to check the effect of reducing the triglyceride (TG) content of preadipocytes according to the extract for each herbal medicine obtained according to Preparation Example 1.

Quantification of intracellular triglycerides was performed by an enzymatic method using a TG determination kit (Asan Pharm Co., Ltd., Korea). Cells were washed 3 times with a phosphate buffer saline (hereafter referred to as PBS), and then lysis buffer (1% Triton X-100 in PBS) was added, followed by sonication performed at 4° C. for 30 seconds and centrifugation (10,000 rpm, 20 min, 4° C.) to separate supernatant. After adding 3 mL of an enzyme solution to 20 μL of the supernatant and reacting at 37° C. for 10 minutes, absorbance was measured at 550 nm. Protein content was measured according to the Bradford method using bovine serum albumin (BSA) as a standard reagent.

Table 1 below shows the results of determining the intracellular triglyceride inhibitory effect of 70% alcohol extract for 100 kinds of herbal medicines.

TABLE 1

| | | TG |
|---|---|---|
| | | inhibitory |
| No. | Herbal extract | effect (%) |
| 1 | Liriopis tuber | 2.4 |
| 2 | Cuscutae semen | 12.4 |
| 3 | Saururus chinensis | 1.6 |
| 4 | Kochia scoparia | −0.4 |
| 5 | Ponciri fructus | 1.6 |
| 6 | Lonicerae flos | 16.3 |
| 7 | Plantaginis semen | 3.7 |
| 8 | Salvia miltiorrhiza | 32.8 |
| 9 | Typhae pollen | 11.4 |
| 10 | Caryophylli flos | 10.6 |
| 11 | Asparagi radix | 9.4 |
| 12 | Psoraleae semen | 12.4 |
| 13 | Chrysanthemi flos | 2.9 |
| 14 | Sophorae flos | −5.1 |
| 15 | Biotae cacumen | 13.4 |
| 16 | Uncaria gambir roxburgh | 3.9 |
| 17 | Ephedrae herba | 1.4 |
| 18 | Corydalis tuber | 11.4 |
| 19 | Zingiberis rhizoma | 6.4 |
| 20 | Artemisiae Apiaceae Herba | 0.7 |
| 21 | Myristicae semen | 6.1 |
| 22 | Ulmus davidiana | −7.4 |
| | var. japonica | |
| 23 | Perillae herba | 13.0 |
| 24 | Cnidium officinale | 5.6 |
| 25 | Portulaca oleracea | 10.8 |
| 26 | Ecliptae herba | 3.5 |
| 27 | Paeonia lactiflora | 38.4 |
| 28 | Zanthoxyli fructus | 1.5 |
| 29 | Anemarrhenae rhizoma | 6.4 |
| 30 | Scutellariae radix | 11.3 |
| 31 | Torilidis fructus | 15.4 |
| 32 | Viticis fructus | −6.4 |
| 33 | Schizonepeta tenuifolia | 2.1 |
| | var. japonica | |
| 34 | Gleditsiae fructus | −3.6 |
| 35 | Magnoliae flos | 8.4 |
| 36 | Angelicae gigantis radix | 10.7 |
| 37 | green tea | 13.6 |
| 38 | Phellodendri cortex | −4.5 |
| 39 | Angelicae dahuricae radix | 18.7 |
| 40 | Akebia quinata | 16.4 |
| 41 | Peucedani radixa | 1.1 |
| 42 | Gastrodiae rhizoma | 9.5 |
| 43 | Chinensis galla | 13.4 |
| 44 | Pinelliae rhizoma | 15.6 |
| 45 | Cynanchum wilfordii mas. | 9.3 |
| | hemsley. | |
| 46 | Castaneae semen | 5.1 |
| 47 | Rehmanniae radix preparata | 11.7 |
| 48 | Mori cotex radicis | 8.7 |
| 49 | Magnoliae cortex | 6.5 |
| 50 | Eriobotrya japonica | 31.4 |
| 51 | Angelicae koreanae radix | 13.5 |
| 52 | Imperata cylindrica | 31.9 |
| | var. koenigii | |
| 53 | Araliae cordatae radix | −4.0 |
| 54 | Foeniculi fructus | 20.1 |
| 55 | Artemisiae capillaris herba | 12.5 |
| 56 | Moutan radicis cortex | 6.9 |
| 57 | Saussureae radix | 13.4 |
| 58 | Polygoni multiflori radix | 7.1 |
| 59 | Betula platyphylla | 2.4 |
| | var. japonica | |
| 60 | Prunellae herba | 13.2 |
| 61 | Eucommiae cortex | 9.6 |
| 62 | Arecae semen | 10.4 |
| 63 | Dryopteris crassirhizoma | 13.1 |
| 64 | Nelumbinis semen | −4.4 |
| 65 | Cinnamon | 6.7 |
| 66 | Stephaniae tetrandrae radix | 16.5 |
| 67 | Acontii tuber | 3.4 |
| 68 | Sanguisorba officinalis | 32.1 |
| 69 | Hoveniae semen cum fructus | 3.5 |
| 70 | Acori graminei rhizoma | 3.0 |
| 71 | Granati pericarpium | 3.6 |
| 72 | Plantago asiatica | 11.9 |
| 73 | Lophatheri herba | −10.9 |
| 74 | Aurantii nobilis pericarpium | 13.5 |
| 75 | Thujae semen | 1.5 |
| 76 | Asteris radix | 1.1 |
| 77 | Caesalpiniae lignum | 15.4 |
| 78 | Cyperi rhizma | 0.3 |
| 79 | Polygalae radix | 2.6 |
| 80 | Armeniacae semen | 5.9 |
| 81 | Rubi fructus | −4.5 |
| 82 | Forsythiae frucus | 13.5 |
| 83 | Glehnia littoralis | 1.6 |
| 84 | Cephalonoplos segetum | 3.7 |
| 85 | Stephaniae tetrandrae radix | 1.6 |
| 86 | Rehmannia glutinosa | 2.4 |
| 87 | Cistanchis herba | 10.6 |
| 88 | Sophorae radix | 4.5 |
| 89 | Schizandrae fructus | 6.9 |
| 90 | Gardeniae fructus | 3.5 |
| 91 | Curcumae longae rhizoma | −7.9 |
| 92 | Erythrinae cortex | −1.4 |
| 93 | Momordica charantia | 9.4 |
| 94 | Lysimachia foenum-graecum | 16.3 |
| 95 | Acanthopanacis cortex | 1.0 |
| 96 | Ledebouriellae radix | 11.4 |
| 97 | Lithospermum erythrorhizon | 2.3 |
| 98 | Houttuyniae herba | 16.5 |
| 99 | Coptidis rhizoma | 2.9 |
| 100 | Solanum nigrum | 13.1 |

Referring to Table 1, among the 100 kinds of herbal medicine extracts, it was found that Salvia miltiorrhiza (no.8), Paeonia lactiflora (no.27), Eriobotrya japonica (no.50), Imperata cylindrica var. koenigii (no.52), and Sanguisorba officinalis (no.68) exhibited excellent effect in inhibiting intracellular triglyceride of 30% or more.

Accordingly, the five kinds of herbal medicines were selected, and effect comparisons were performed on mixed extracts by weight of each herbal medicine.

Table 2 below shows results of determining the intracellular triglyceride inhibitory effect of the extracts according to each alcohol concentration (0, 20, 50, 70, and 100%) for each of Salvia miltiorrhiza, Paeonia lactijlora, Eriobotrya japonica, Imperata cylindrica var. koenigii, and Sanguisorba Officinalis selected according to Experimental Example 1.

TABLE 2

| | | | | Imperata | | | | |
|---|---|---|---|---|---|---|---|---|
| | | | | cylindrica | | | | TG |
| | Salvia | Paeonia | Eriobotrya | var. | Sanguisorba | Alcohol | | inhibitory |
| Preparation | miltiorrhiza | lactiflora | japonica | koenigii | officinalis | concentration | Yield | effect |
| Example | % (w/w) | % (w/w) | % (w/w) | % (w/w) | % (w/w) | (%) | (%) | (%) |
| Comparative | 100 | 0 | 0 | 0 | 0 | 0 | 21.9 | 19.5 |
| Example 1 | | | | | | | | |
| Comparative | 0 | 100 | 0 | 0 | 0 | 0 | 11.6 | 16.1 |
| Example 2 | | | | | | | | |
| Comparative | 0 | 0 | 100 | 0 | 0 | 0 | 11.6 | 13.6 |
| Example 3 | | | | | | | | |
| Comparative | 0 | 0 | 0 | 100 | 0 | 0 | 4.1 | 14.7 |
| Example 4 | | | | | | | | |
| Comparative | 0 | 0 | 0 | 0 | 100 | 0 | 24.1 | 13.8 |
| Example 5 | | | | | | | | |
| Comparative | 100 | 0 | 0 | 0 | 0 | 20 | 22.6 | 26.7 |
| Example 6 | | | | | | | | |
| Comparative | 0 | 100 | 0 | 0 | 0 | 20 | 12 | 26.5 |
| Example 7 | | | | | | | | |
| Comparative | 0 | 0 | 100 | 0 | 0 | 20 | 10.5 | 21.0 |
| Example 8 | | | | | | | | |
| Comparative | 0 | 0 | 0 | 100 | 0 | 20 | 4.5 | 19.6 |
| Example 9 | | | | | | | | |
| Comparative | 0 | 0 | 0 | 0 | 100 | 20 | 22.3 | 21.4 |
| Example 10 | | | | | | | | |
| Comparative | 100 | 0 | 0 | 0 | 0 | 50 | 21.4 | 28.4 |
| Example 11 | | | | | | | | |
| Comparative | 0 | 100 | 0 | 0 | 0 | 50 | 10.8 | 28.0 |
| Example 12 | | | | | | | | |
| Comparative | 0 | 0 | 100 | 0 | 0 | 50 | 15.6 | 21.6 |
| Example 13 | | | | | | | | |
| Comparative | 0 | 0 | 0 | 100 | 0 | 50 | 5.4 | 20.9 |
| Example 14 | | | | | | | | |
| Comparative | 0 | 0 | 0 | 0 | 100 | 50 | 22.9 | 25.4 |
| Example 15 | | | | | | | | |
| Comparative | 100 | 0 | 0 | 0 | 0 | 70 | 24.1 | 39.4 |
| Example 16 | | | | | | | | |
| Comparative | 0 | 100 | 0 | 0 | 0 | 70 | 12.4 | 38.0 |
| Example 17 | | | | | | | | |
| Comparative | 0 | 0 | 100 | 0 | 0 | 70 | 17.2 | 32.6 |
| Example 18 | | | | | | | | |
| Comparative | 0 | 0 | 0 | 100 | 0 | 70 | 6.12 | 30.4 |
| Example 19 | | | | | | | | |
| Comparative | 0 | 0 | 0 | 0 | 100 | 70 | 25.4 | 33.8 |
| Example 20 | | | | | | | | |
| Comparative | 100 | 0 | 0 | 0 | 0 | 100 | 21.3 | 29.4 |
| Example 21 | | | | | | | | |
| Comparative | 0 | 100 | 0 | 0 | 0 | 100 | 10.4 | 26.1 |
| Example 22 | | | | | | | | |
| Comparative | 0 | 0 | 100 | 0 | 0 | 100 | 15.6 | 21.7 |
| Example 23 | | | | | | | | |
| Comparative | 0 | 0 | 0 | 100 | 0 | 100 | 5.2 | 23.0 |
| Example 24 | | | | | | | | |
| Comparative | 0 | 0 | 0 | 0 | 100 | 100 | 20.3 | 20.1 |
| Example 25 | | | | | | | | |

Referring to Table 2, in the case of single extracts of Salvia miltiorrhiza, Paeonia lactijlora, Eriobotrya japonica, Imperata cylindrica var. koenigii, and Sanguisorba officinalis, the extraction yields were the highest at the 70% alcohol concentration condition. In addition, it was confirmed that the triglyceride inhibitory effect in preadipocytes was also the most excellent in the condition of 70% alcohol (Comparative Examples 16 to 20).

Accordingly, the alcohol concentration was set to 70%, and comparisons of triglyceride inhibitory effect under mixed extraction conditions for each herbal medicine were performed.

Table 3 below shows results of determining the triglyceride inhibitory effect according to the mixed extractions of each herbal medicine at 70% alcohol concentration.

TABLE 3

| | | | | Imperata | | | | |
|---|---|---|---|---|---|---|---|---|
| | | | | cylindrica | | | | TG |
| | Salvia | Paeonia | Eriobotiya | var. | Sanguisorba | Alcohol | | inhibitory |
| Preparation | miltiorrhiza | lactiflora | japonica | koenigii | officinalis | concentration | Yield | effect |
| Example | % (w/w) | % (w/w) | % (w/w) | % (w/w) | % (w/w) | (%) | (%) | (%) |
| Reference | 50 | 50 | 0 | 0 | 0 | 70 | 18.9 | 82.6 |
| Example 1 | | | | | | | | |
| Reference | 50 | 0 | 50 | 0 | 0 | 70 | 16.4 | 29.4 |
| Example 2 | | | | | | | | |
| Reference | 50 | 0 | 0 | 50 | 0 | 70 | 17.0 | 31.0 |
| Example 3 | | | | | | | | |
| Reference | 50 | 0 | 0 | 0 | 50 | 70 | 16.2 | 30.7 |
| Example 4 | | | | | | | | |
| Reference | 0 | 50 | 50 | 0 | 0 | 70 | 13.4 | 20.7 |
| Example 5 | | | | | | | | |
| Reference | 0 | 50 | 0 | 50 | 0 | 70 | 8.9 | 31.4 |
| Example 6 | | | | | | | | |
| Reference | 0 | 50 | 0 | 0 | 50 | 70 | 15.1 | 20.9 |
| Example 7 | | | | | | | | |
| Reference | 0 | 0 | 50 | 50 | 0 | 70 | 9.4 | 26.4 |
| Example 8 | | | | | | | | |
| Reference | 0 | 0 | 50 | 0 | 50 | 70 | 20.1 | 24.5 |
| Example 9 | | | | | | | | |
| Reference | 0 | 0 | 0 | 50 | 50 | 70 | 15.1 | 20.1 |
| Example 10 | | | | | | | | |
| Reference | 33.3 | 33.3 | 33.3 | 0 | 0 | 70 | 16.1 | 46.8 |
| Example 11 | | | | | | | | |
| Reference | 33.3 | 33.3 | 0 | 33.3 | 0 | 70 | 12.9 | 43.8 |
| Example 12 | | | | | | | | |
| Reference | 33.3 | 33.3 | 0 | 0 | 33.3 | 70 | 15.0 | 51.7 |
| Example 13 | | | | | | | | |
| Reference | 33.3 | 0 | 33.3 | 33.3 | 0 | 70 | 13.7 | 30.4 |
| Example 14 | | | | | | | | |
| Reference | 33.3 | 0 | 33.3 | 0 | 33.3 | 70 | 11.6 | 31.6 |
| Example 15 | | | | | | | | |
| Reference | 33.3 | 0 | 0 | 33.3 | 33.3 | 70 | 10.4 | 27.1 |
| Example 16 | | | | | | | | |
| Reference | 0 | 33.3 | 33.3 | 33.3 | 0 | 70 | 9.8 | 26.0 |
| Example 17 | | | | | | | | |
| Reference | 0 | 33.3 | 33.3 | 0 | 33.3 | 70 | 11.6 | 24.1 |
| Example 18 | | | | | | | | |
| Reference | 0 | 33.3 | 0 | 33.3 | 33.3 | 70 | 16.4 | 30.8 |
| Example 19 | | | | | | | | |
| Reference | 0 | 0 | 33.3 | 33.3 | 33.3 | 70 | 14.1 | 21.0 |
| Example 20 | | | | | | | | |
| Reference | 25 | 25 | 25 | 25 | 0 | 70 | 15.1 | 41.3 |
| Example 21 | | | | | | | | |
| Reference | 25 | 0 | 25 | 25 | 25 | 70 | 14.6 | 30.9 |
| Example 22 | | | | | | | | |
| Reference | 25 | 25 | 0 | 25 | 25 | 70 | 13.0 | 40.6 |
| Example 23 | | | | | | | | |
| Reference | 25 | 25 | 25 | 0 | 25 | 70 | 18.1 | 41.7 |
| Example 24 | | | | | | | | |
| Reference | 25 | 25 | 25 | 25 | 0 | 70 | 12.1 | 41.9 |
| Example 25 | | | | | | | | |
| Reference | 20 | 20 | 20 | 20 | 20 | 70 | 15.4 | 39.4 |
| Example 26 | | | | | | | | |

Referring to Table 3, it was found that the triglyceride inhibitory effect was the most excellent in the extract from a mixture of Salvia miltiorrhiza and Paeonia lactiflora (Reference Example 1) at a weight ratio of 1:1 among the mixed extracts by weight for the five kinds of herbal medicines. It was confirmed that the mixed extract of Salvia miltiorrhiza and Paeonia lactiflora had a significantly superior inhibitory effect compared to the extracts from mixtures of other medicinal herbs at different weight ratios.

Accordingly, effect comparison tests were performed according to a ratio of the mixed extract of Salvia miltiorrhiza and Paeonia lactiflora.

### <Experimental Example 2> Determination of the Inhibitory Effect on adipocytes Differentiation According to the Medicinal Herb Complex Extracts

Experiments were performed to determine the inhibitory effect on adipocytes differentiation of mixed extracts by weight of Salvia miltiorrhiza and Paeonia lactiflora selected in Experimental Example 1.

Cells used in the experiment were 3T3—L1 preadipocytes, which were cultured in DMEM (10% FBS, 100 unit/mL of penicillin G sodium, 100 μg/mL of streptomycin sulfate) in an incubator at 5% CO₂ and 37° C. When reaching 100% confluent, differentiation was induced for 2 days by exchanging the medium for DMEM containing 0.5 mM IBMX, 1 μM DEX, and differentiation inducers of insulin of 1 μg/mL, and thereafter, it was exchanged for DMEM containing 1 μg/mL insulin every 2 days. The cells after 10 days of differentiation were used for analysis. 3T3—L1 preadipocytes were treated with 0.5 mM IBMX, 1 μM DEX, and 1 μg/mL insulin to induce differentiation as a control group, and each extract was treated for 2 days to induce differentiation as a test group.

Oil red O staining was performed to check the generation of adipocytes in the differentiated cells. The cultured cells were washed with PBS, fixed with 3.7% formalin for 10 minutes, washed with deionized water, treated with Oil red O, and then stained at room temperature for 30 minutes. After that, the staining solution was removed, the cells were washed 3 times with deionized water and dimethyl sulfoxide (DMSO) was added to the stained cells to extract fat, and then absorbance was measured at 510 nm using ELISA (Molecular Devices, USA). In addition, the intracellular triglyceride inhibitory effect was determined in the same manner as in Experimental Example 1.

Table 4 below shows results of checking the inhibitory effects on adipocytes differentiation of the mixed extracts by weight of Salvia miltiorrhiza and Paeonia lactiflora according to Experimental Example 2.

TABLE 4

| | | | | | | Fat |
|---|---|---|---|---|---|---|
| | | | | | TG | differentiation |
| | Salvia | Paeonia | Alcohol | | inhibitory | inhibitory |
| Preparation | miltiorrhiza | lactiflora | concentration | Yield | effect | effect |
| Example | % (w/w) | % (w/w) | (%) | (%) | (%) | (%) |
| Example 1 | 9.1 | 90.9 | 70 | 5.1 | 41.2 | 40.6 |
| Example 2 | 16.7 | 83.3 | 70 | 6.8 | 38.4 | 40.0 |
| Example 3 | 20 | 80 | 70 | 9.6 | 40.6 | 39.1 |
| Example 4 | 25 | 75 | 70 | 12.4 | 49.5 | 45.7 |
| Example 5 | 33.3 | 66.7 | 70 | 13.4 | 52.9 | 50.9 |
| Example 6 | 50 | 50 | 70 | 18.9 | 89.2 | 82.3 |
| Example 7 | 66.7 | 33.3 | 70 | 15.4 | 50.1 | 46.3 |
| Example 8 | 75 | 25 | 70 | 16.4 | 49.5 | 40.3 |
| Example 9 | 80 | 20 | 70 | 18.4 | 43.1 | 40.7 |
| Example 10 | 83.3 | 16.7 | 70 | 20.1 | 40.3 | 38.4 |
| Example 11 | 90.9 | 9.1 | 70 | 22.4 | 40.8 | 47.5 |

Referring to Table 4, it was found that the triglycerides inhibitory and fat differentiation inhibitory effects were excellent in the mixed extracts by weight ratio of Salvia miltiorrhiza and Paeonia lactiflora. In particular, it was found that the effects were the most excellent in a weight ratio of 1:1 in Example 6.

### <Experimental Example 3> Determination of Fat Accumulation Inhibitory Effect in Hepatocytes of Salvia Miltiorrhiza Extract, Paeonia Lactiflora Extract, or Mixed Extract of Salvia Miltiorrhiza and Paeonia Lactiflora

In order to check the amount of triglycerides in hepatocyte HepG2 cells for each extract of the single extract of Salvia miltiorrhiza of Comparative Example 16 of Table 2, the single extract of Paeonia lactiflora of Comparative Example 17, and the mixed extracts by weight ratio of Salvia miltiorrhiza and Paeonia lactiflora of Examples 1 to 11 of Table 4, it was analyzed by staining with Oil Red O suitable for measuring intracytoplasmic lipids.

HepG2 cells were seeded in a 24-well culture plate at a concentration of 1 x10⁵ cells/well, and cultured until the cell density reached 80%, and then to induce fat accumulation in a state in which further growth of cells was suppressed, the culture medium was replaced with a medium containing no fetal bovine serum-free medium and 2% bovine serum albumin (BSA) and the cells were cultured overnight. After removing the culture medium, to induce fat accumulation, sodium palmitate-fatty acid-free BSA conjugation complex was treated with cell culture medium containing palmitate and Salvia miltiorrhiza and Paeonia lactiflora extracts at concentrations of 100 and 200 μg/ml.

After 24 hours of treatment, it was washed twice with DPBS (Dulbecco's phosphate-buffered saline), fixed with paraformaldehyde solution for 10 minutes, washed with 60% isopropanol, and then dyed with Oil Red O dye for 30 minutes. After washing several times with distilled water, the absorbance was measured at 500 nm by eluting with isopropanol to analyze the degree of dyeing. As a positive control, omega 3 was used at a concentration of 200 μg/ml.

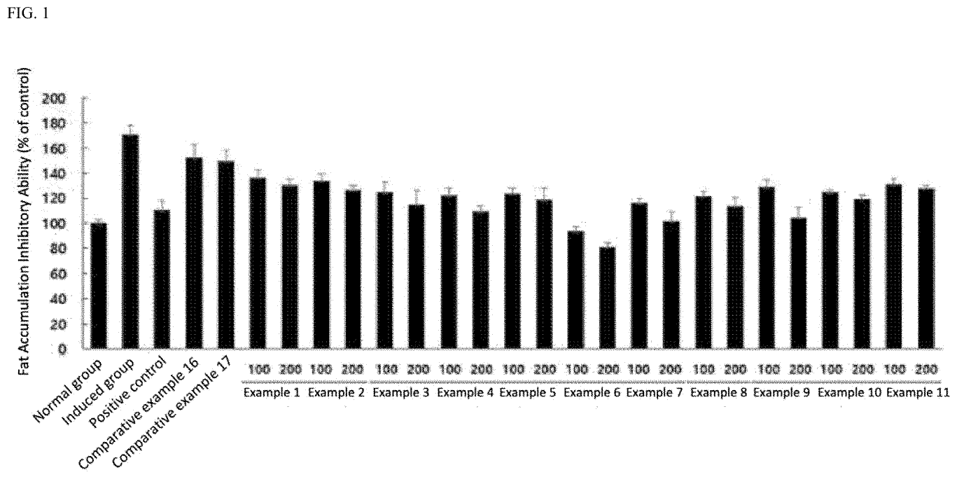

FIG. 1 is a graph showing the effect of inhibiting the accumulation of fat in hepatocytes according to the treatment with a Salvia miltiorrhiza extract or a Paeonia lactiflora extract, or mixed extracts of Salvia miltiorrhiza and Paeonia lactiflora according to an experimental example of the present invention.

Referring to FIG. 1, in the case of treating with Examples 1 to 11, which are extracts from mixtures of weight ratios of Salvia miltiorrhiza and Paeonia lactiflora, it was found that triglycerides production was inhibited in a concentration-dependent manner in hepatocytes compared to Comparative Examples 16 and 17, which are the Salvia miltiorrhiza extract and Paeonia lactiflora extract, respectively, and in particular, it was confirmed that the effect was the most excellent in Example 6.

### <Experimental Example 4> Confirmation of Blood Triglycerides Reduction Effect in Animal Experiments of Salvia Miltiorrhiza or Paeonia Lactiflora Extract or Mixed Extract of Salvia Miltiorrhiza and Paeonia Lactiflora

The blood triglycerides reduction effect was confirmed through animal experiments on each extract of the single extract of Salvia miltiorrhiza of Comparative Example 16 of Table 2, the single extract of Paeonia lactiflora of Comparative Example 17, and the mixed extracts of by weight ratios of Salvia miltiorrhiza and Paeonia lactiflora of Examples 1 to 11 of Table 4.

As a positive control, omega-3 fatty acid was used. Specifically, after 10 weeks of high-fat diet using C57BL/6 APOE-/-mice, the extracts were administered daily at a concentration of 400 mg/kg for 7 days. On the day of autopsy, about 500 μL of blood collected from the abdominal vena cava from all animals was collected, placed in a tube without anticoagulants, left for 90 minutes, and centrifuged (3,000 rpm, 10 minutes, room temperature), and triglycerides (TG) were measured with the obtained serum using a blood biochemical analyzer (BS220, Mindray).

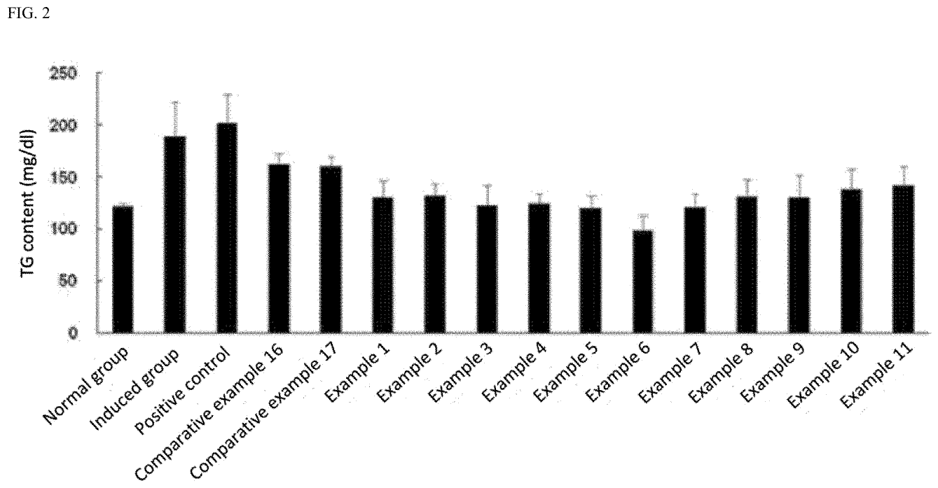

FIG. 2 is a graph showing changes in triglyceride (TG) content according to treatment with a Salvia miltiorrhiza or Paeonia lactiflora extract, or mixed extracts of Salvia miltiorrhiza and Paeonia lactiflora, according to an experimental example of the present invention.

Referring to FIG. 2, it was found that the TG reduction effect was better in the case of treating with Examples 1 to 11, which are mixed extracts by weight ratios of Salvia miltiorrhiza and Paeonia lactiflora, compared to Comparative Examples 16 and 17, which are single extracts of Salvia miltiorrhiza and Paeonia lactiflora, respectively. They showed superior effects than the omega-3 fatty acid used as a positive control. From the above results, it was possible to confirm the pharmacologically active synergistic effect of the mixed extracts according to the present invention.

### <Experimental Example 5> Confirmation of Blood Cholesterol Alleviation Effect in Animal Experiments of Salvia Miltiorrhiza or Paeonia Lactiflora Extract or Mixed Extract of Salvia Miltiorrhiza and Paeonia Lactiflora

The effect of reducing blood cholesterol was confirmed through animal experiments on the single extract of Salvia miltiorrhiza of Comparative Example 16, the single extract of Paeonia lactiflora of Comparative Example 17, and the mixed extracts by weight ratios of Salvia miltiorrhiza and Paeonia lactiflora of Examples 1 to 11.

As a positive control, omega-3 fatty acid was used. Specifically, after 10 weeks of high-fat diet using C57BL/6 APOE-/-mice, the extracts were administered daily at a concentration of 400 mg/kg for 7 days. On the day of autopsy, about 500 μL of blood collected from the abdominal vena cava from all animals was collected, placed in an anticoagulant (EDTA-2K) tube, and centrifuged (3,000 rpm, 10 minutes, room temperature) to separate plasma, and total cholesterol content, LDL cholesterol, and HDL cholesterol content were measured.

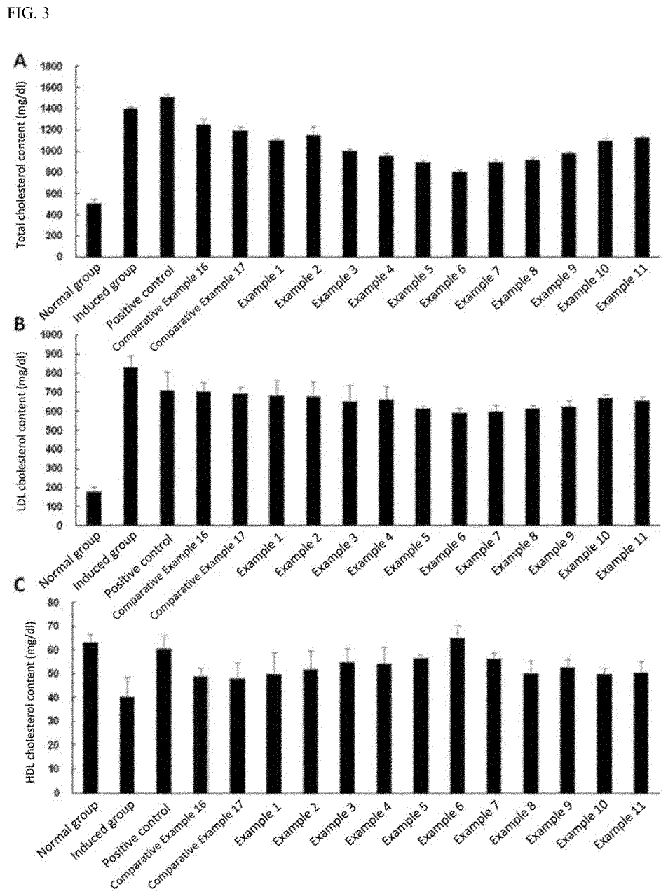

FIG. 3 shows graphs showing changes in cholesterol content according to treatment with a Salvia miltiorrhiza or Paeonia lactiflora extract, or mixed extracts of Salvia miltiorrhiza and Paeonia lactiflora according to an experimental example of the present invention, wherein A shows total cholesterol content, B shows low-density lipoprotein (LDL) cholesterol content, and C shows high-density lipoprotein (HDL) cholesterol content.

Referring to FIG. 3, when treating with Examples 1 to 11, which are mixed extracts by weight ratios of Salvia miltiorrhiza and Paeonia lactiflora, it was found that total cholesterol content and LDL cholesterol content were reduced and the HDL cholesterol content increased compared to Comparative Examples 16 and 17, which are single extracts of Salvia miltiorrhiza and Paeonia lactiflora, respectively. They showed a superior effect than the omega-3 fatty acid used as a positive control. From the above results, it was possible to confirm the pharmacologically active synergistic effect of the mixed extracts according to the present invention.

### <Experimental Example 6> Confirmation of the Effect of Reducing Fatty Liver and Visceral Fat in Animal Experiments of the Mixed Extracts

Histopathological examination was performed for the single extract of Salvia miltiorrhiza of Comparative Example 16 of Table 2, the single extract of Paeonia lactiflora of Comparative Example 17, and the mixed extract of Salvia miltiorrhiza and Paeonia lactiflora of Example 6 of Table 4 above.

As a positive control, omega-3 fatty acid was used. Specifically, after 10 weeks of high-fat diet using C57BL/6 APOE-/-mice, Comparative Examples 16, 17, and Example 6 were administered daily at a concentration of 400 mg/kg for 7 days. On the day of autopsy, liver and abdominal fat (white adipose tissue) were extracted from all animals and fixed with 10% neutral formalin. Tissue sections were prepared through general tissue processing such as dehydrated paraffin and paraffin embedding of tissue of the fixed organ tissue. Samples were prepared through the sectioning process and evaluated by performing hematoxylin & eosin staining.

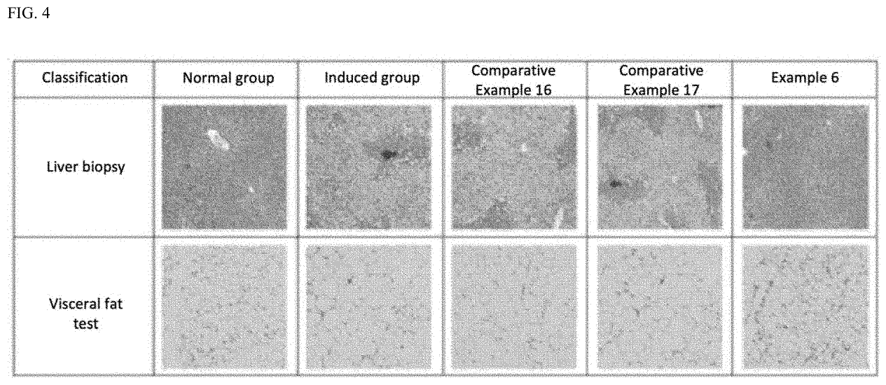

FIG. 4 shows histopathological examination results of a mixed extract according to an experimental example of the present invention.

Referring to FIG. 4, when comparing the fat lesions and fat accumulation in the liver according to the high-fat diets of Comparative Examples 16 and 17 and Example 6, it was found that the fat accumulation in the liver was inhibited in Example 6. In addition, it was confirmed that the size of adipocytes of white fat in the abdominal fat increased in the high-fat diet and decreased in Example 6, thereby affecting the production of visceral fat.

Through the above series of results, when the mixed extract of Salvia miltiorrhiza and Paeonia lactiflora of the present invention was used as an active ingredient, it was possible to confirm the effect of inhibiting adipocytes differentiation and triglycerides accumulation in vitro. In addition, in vivo, it was confirmed that blood triglyceride, total cholesterol content, LDL cholesterol, and HDL cholesterol content were improved according to each of the extracts, and also it was confirmed that the extracts exhibited the effect of inhibiting fatty liver and visceral fat formation.

Specific parts of the present invention have been described above in detail, and it is clear to those skilled in the art that these specific descriptions are merely preferred embodiments and the scope of the present invention is not limited thereby. Therefore, the substantial scope of the present invention is defined by the appended claims and their equivalents.

What is claimed is:

1. A pharmaceutical composition for prevention or treatment of a lipid metabolism disorder, the pharmaceutical composition comprising a Salvia miltiorrhiza extract, a Paeonia lactiflora extract, or a mixed extract of Salvia miltiorrhiza and Paeonia lactiflora as an active ingredient.

2. The pharmaceutical composition according to claim 1, wherein the extract is extracted with anyone solvent selected from the group consisting of water, alcohols having 1 to 4 carbon atoms, and mixed solvents thereof

3. The pharmaceutical composition according to claim 1, wherein the extract is extracted with a 60 to 80 wt % ethanol aqueous solution.

4. The pharmaceutical composition according to claim 1, wherein the mixed extract is extracted from a mixture of Salvia miltiorrhiza and Paeonia lactiflora in a weight ratio of (0.1 to 10):1.

5. The pharmaceutical composition according to claim 1, wherein the mixed extract is extracted from a mixture of Salvia miltiorrhiza and Paeonia lactiflora in a weight ratio of 1:1 with a 70 wt % ethanol aqueous solution.

6. The pharmaceutical composition according to claim 1, wherein the composition inhibits differentiation of preadipocytes into adipocytes.

7. The pharmaceutical composition according to claim 1, wherein the composition reduces blood triglycerides or blood cholesterol.

8. The pharmaceutical composition according to claim 1, wherein the lipid metabolism disorder is one or more selected from the group consisting of dyslipidemia including hypertriglyceridemia, hypercholesterolemia, or hyperlipidemia, fatty liver, and obesity.

9. A health functional food composition for prevention or alleviation of a lipid metabolism disorder, the health functional food composition comprising Salvia miltiorrhiza extract, a Paeonia lactiflora extract, or a mixed extract of Salvia miltiorrhiza and Paeonia lactiflora as an active ingredient.

10. The health functional food composition according to claim 9, wherein the mixed extract is extracted from a mixture of Salvia miltiorrhiza and Paeonia lactiflora in a weight ratio of 1:1 with a 70 wt % ethanol aqueous solution.

11. A health functional food composition for alleviation of blood lipids comprising a Salvia miltiorrhiza extract, a Paeonia lactiflora extract, or a mixed extract of Salvia miltiorrhiza and Paeonia lactiflora, as an active ingredient.
